(19) United States
(12) Patent Application Publication
(10) Pub. No.: US 2016/0120877 A1
(43) Pub. Date: May 5, 2016

(54) PHARMACEUTICAL COMPOSITIONS FOR SUSTAINED DELIVERY OF BENZODIAZEPINE ANTAGONISTS

(71) Applicants: James S Rowe, San Souci (AU); Grau Consoni, San Souci (AU)

(72) Inventors: James S Rowe, San Souci (AU); Grau Consoni, San Souci (AU)

(21) Appl. No.: 14/890,761

(22) PCT Filed: Jul. 15, 2011
(86) PCT No.: PCT/AU2011/000897

§ 371 Date: Nov. 12, 2015

(30) Foreign Application Priority Data
Jul. 16, 2010 (AU) 2010903183

(51) Int. Cl. A61K 31/5517; A61K 47/34; A61K 9/00

(52) U.S. Cl. CPC A61K 31/5517; A61K 9/0024; A61K 47/34

(57) ABSTRACT

The present invention relates to sustained release formulations of benzodiazepine antagonists and their use.

### FIELD OF THE INVENTION

The present invention relates to sustained release formulations of benzodiazepine antagonists and in particular to sustained release formulations of flumazenil.

The invention has been developed primarily for use as pharmaceutical composition for sustained release of benzodiazepine antagonists and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Benzodiazepines are psychoactive drugs that enhance the effect of the neurotransmitter gamma-aminobutyric acid (GABA) by binding to the GABA receptor and increasing or enhancing GABA-mediated neurotransmission, i.e., the conductance of chloride ions through the GABA_{A} receptor channel (Ashton 2002 “Benzodiazepines: How They Work and How to Withdraw”, Newcastle upon Tyne: School of Neurosciences, Division of Psychiatry).

Benzodiazepines have sedative, hypnotic, anxiolytic, anticonvulsant, muscle relaxing and amnesic actions and are used for anaesthesia and the treatment of anxiety, panic, seizures, agitation and insomnia (Page et al 2002, Integrated Pharmacology (2^{nd} edition), Mosby; Olkkola & Ahonen 2008, Handb Exp Phamacol 182: 335-360).

Combining benzodiazepines with alcohol, opiates or tricyclic antidepressants markedly increases the toxicity of benzodiazepines and may result in benzodiazepine overdose. Benzodiazepine overdose may lead to drowsiness, slurred speech, nystagmus, hypotension, ataxia, coma, respiratory depression and cardiorespiratory arrest (White & Irvine 1999 Addiciton 94(7): 961-972; Ramrakha & Moore 2004 “Chapter 14: Drug overdoses”, Oxford Handbook of Acute Medicine (2^{nd} edition), Oxford University Press, pp 791-838).

Benzodiazepine antagonists reverse the effects of benzodiazepines by competitively inhibiting benzodiazepine binding to the GABA receptor. Benzodiazepine antagonists are used for a complete or partial reversal of the effects of benzodiazepines and for the management of benzodiazepine overdose (Anexate®, Medical Information Management System (MIMS) Abbreviated Prescribing Information, Sydney 2010).

Flumazenil (ethyl 8-fluoro-5,6-dihydro-5-methyl-6-oxo-4H-imidazo[1,5-a](1,4)benzodiazepine-3-carboxylate) is benzodiazepine antagonist that has been found to be effective in the complete or partial reversal of the sedative effects of benzodiazepines (e.g., in cases where general anaesthesia has been induced and/or maintained with benzodiazepines, where sedation has been produced with benzodiazepines for diagnostic and therapeutic procedures) and for the treatment of benzodiazepine overdose. Flumazenil has also been shown to be effective in the treatment of sleepiness (WO 2009/114740), drug addiction (WO 2009/027564), nervous system disorders (WO 2009/027564), psychiatric conditions (WO 2009/016329), Down Syndrome (WO 2008/128116), depressive disorders (WO 2008/006918), memory deficits (WO 2007/018660), alcohol and/or stimulant substance abuse (WO 2006/115743), neuropathic pain (WO 2006/115302), cocaine dependence (WO 2002/064213), and alcohol dependence (WO 2002/056964).

Flumazenil has also been shown to be beneficial in the treatment of methamphetamine dependence. Studies conducted in adult male rats found that 5 weeks of methamphetamine (3 mg/kg) treatment produced a 4- to 5-fold increase in GABA receptor subunit α4 expression and that, during methamphetamine withdrawal, flumazenil (10 mg/kg) reduced α4 levels to control levels in the hippocampus and cortex (Sanna et al. 2003. J Neurosci 23(37): 11711-11724. Clinical trials have shown that treatment with flumazenil (along with, hydroxyzine and gabapentin) substantially reduce methamphetamine cravings (Urschel et al 2007, Mayo Clinic Proceedings 82(10): 1170-1178).

Flumazenil has an imidazobenzodiazepine structure, a calculated molecular weight of 303.3 and the following structural formula:

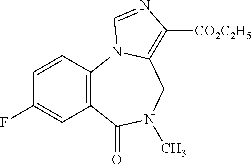 [SMILES: CCOC(=O)c1ncn2c1CN(C)C(=O)c1cc(F)ccc1-2]

Administration of flumazenil is primarily by intravenous injection. However, as flumazenil has a relatively short terminal half-life (i.e., 40 to 80 minutes), repeat (e.g., daily) infusions of flumazenil are often required—necessitating extended periods of hospitalisation and hampering treatment compliance.

It is an object of the present invention to overcome or ameliorate at least one of the disadvantages of the prior art, or to provide a useful alternative.

It is an object of the invention in its preferred form to provide a pharmaceutical composition for the sustained release of benzodiazepine antagonists.

### SUMMARY OF THE INVENTION

Surprisingly, the pharmaceutical composition of the present invention provides a simple, stable formulation for the sustained release of benzodiazepine antagonists that avoids the requirement for frequent or lengthy injections/infusions.

According to a one aspect, the present invention provides a sustained release pharmaceutical composition comprising a benzodiazepine antagonist and a biocompatible excipient.

The biocompatible excipient may be a polymer that is able to form a gel-like depot.

According to another aspect, the present invention provides a sustained release pharmaceutical composition comprising a benzodiazepine antagonist and a polymer that is able to form a gel-like depot.

According to another aspect, the present invention relates to a sustained release pharmaceutical composition comprising a benzodiazepine antagonist and a polymer that is able to form a gel-like depot, wherein the composition has the following in vitro dissolution rate when measured using a Vankel VK 7000 dissolution apparatus set at 37° C. and 20 rpm, a dialysate of 800 ml of phosphate buffer pH 4.0 and dialysis tubing that retains >90% of cytocrome C (M.W. 12.400) in solution over a 10 hrs period:
between 10 and 30% of benzodiazepine antagonist released after 6 hours,
between 20 and 40% of benzodiazepine antagonist released after 24 hours,
between 30 and 50% of benzodiazepine antagonist released after 2 days,
between 40 and 60% of benzodiazepine antagonist released after 3 days,
at least 60% of benzodiazepine antagonist released after 7 days,
at least 70% of benzodiazepine antagonist released after 10 days,
at least 80% of benzodiazepine antagonist released after 14 days.

The polymer may be selected from the group consisting of polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, polymalic acids, polyamino acids, polyvinylpyrrolidones, polyethylene glycols, polyhydroxycelluloses, chitins, chitosans and polyorthoesters, and copolymers, terpolymers and combinations and mixtures thereof. Preferably the polymer is selected from the group consisting of polylactides and copolymers thereof with glycolide.

The benzodiazepine antagonist may be flumazenil.

The composition may comprise flumazenil and a copolymer of 3,6-dimethyl-1,4-dioxane-2,5-dione and 1,4-dioxane-2,5-dione.

The composition may further comprise a water-miscible pharmaceutically acceptable organic solvent. The organic solvent may be selected from the group consisting of N-methyl-2-pyrrolidone, 2-pyrrolidone, dimethyl sulfoxide, glycerol formal, ethanol, propylene glycol and combination and mixtures thereof.

The composition may comprise flumazenil, a copolymer of 3,6-dimethyl-1,4-dioxane-2,5-dione and 1,4-dioxane-2,5-dione, and N-methyl-2-pyrrolidone.

The composition are preferably adapted for non-systemic administration, for example intramuscular, intradermal or subcutaneous administration or implantation.

Administration or implantation may be by injection.

The composition may be in liquid form and be able to form a gel-like depot upon for intramuscular, intradermal or subcutaneous injection.

According to another aspect, the present invention provides a method for treating a disease or disorder, an addiction or dependence, or an overdose in a subject comprising administering to the subject a composition according to the invention.

According to another aspect, the present invention provides use of a composition according to the invention for the manufacture of a medicament for treating a disease or disorder, an addiction or dependence, or an overdose.

The disease or disorder may be selected from the group consisting of sleepiness, a nervous system disorder, a psychiatric condition, Down Syndrome, a depressive disorder, memory deficit and neuropathic pain.

The addiction or dependence may be selected from the group consisting of drug addiction, alcohol and/or stimulant substance abuse, cocaine dependence, methamphetamine dependence and alcohol dependence.

The overdose may be benzodiazepine overdose.

According to another aspect, the present invention provides a method of maintaining a therapeutically effective blood level of a benzodiazepine antagonist in a subject for a period of at least one day comprising non-systemic administration to the subject of a single dose of a composition according to the invention.

The period may be at least 4 days, at least 8 days, at least 12 days or at least 14 days.

The therapeutically effective blood level may be at least 1 ng/ml benzodiazepine antagonist in blood.

According to another aspect, the present invention provides a method of maintaining a therapeutically effective blood level of a benzodiazepine antagonist in a subject for a period of at least one day comprising non-systemic administration to the subject of a single dose of a composition according to the invention, wherein the blood level is:

| Time post | Benzodiazepine |
|---|---|
| administration | antagonist in blood |
| (days) | (ng/ml) |
| 1 | 2.6 to 3.2 |
| 4 | 2.2 to 2.8 |
| 6 | 2.8 to 3.6 |
| 9 | 2.9 to 3.7 |
| 12 | 1.8 to 2.2 |
| 14 | 1.3 to 1.7 |

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The skilled addressee would appreciate that the term “benzodiazepine antagonist” includes within its scope benzodiazepine receptor antagonists, GABA benzodiazepine binding site antagonists and compounds having an imidazobenzodiazepine structure (e.g., imidazobenzodiazepine derivatives.

Benzodiazepine antagonists include, for example, flumazenil, ethyl-5-isopropoxy-4-methyl-beta-carboline-3-carboxylate and the compounds disclosed in U.S. Pat. No. 4,316,839, U.S. Pat. No. 4,352,815, U.S. Pat. No. 4,346,030, U.S. Pat. No. 4,489,003, U.S. Pat. No. 4,616,010 and U.S. Pat. No. 4,775,671 (herein incorporated by reference).

Benzodiazepine antagonists may be used for the treatment of benzodiazepine overdose, sleepiness, drug addiction, nervous system disorders, psychiatric conditions, Down Syndrome, depressive disorders, memory deficits, alcohol and/or stimulant substance abuse, neuropathic pain, cocaine dependence, methamphetamine dependence or alcohol dependence.

The skilled addressee would appreciate that the term “addiction” includes within its scope substance abuse and substance dependence.

### BRIEF DESCRIPTION OF THE FIGURES

FIG. 1: Effect of polymer on flumazenil release

FIG. 2: Effect of autoclaving on flumazenil release

FIG. 3: Effect of polymer concentration on flumazenil release

FIG. 4: Flumazenil release over 14 days in vitro.

FIG. 5: Flumazenil release over 14 days in vivo.

### DETAILED DESCRIPTION OF THE INVENTION

Preferred embodiments of the invention will now be described, by way of example only.

This invention is based on the surprising finding that combining a benzodiazepine antagonist with a polymer forms a sustained release formulation that, when implanted in a subject, maintains therapeutic levels (i.e., greater than 1 ng/ml) of the benzodiazepine antagonist in blood for up to 14 days. In contrast, intravenous administration of the benzodiazepine antagonist alone results in therapeutic levels of the benzodiazepine antagonist for only a matter of minutes and, therefore, constant infusion is required.

The chosen polymer system results in a benzodiazepine antagonist formulation that is normally present and administered in a liquid form but forms a gel-like depot in vivo. The term “gel-like”, as used in the context of the present invention, is intended to encompass any higher viscosity or density state when compared to a liquid state, e.g., a semi-solid or solid state. The term “depot”, as used in the present invention, is intended to encompass any form of localised deposit of an active agent wherein the active agent is gradually released from the localised deposit. Thus, in the context of the present invention, the benzodiazepine antagonist formulation is able to change its physical state from a liquid to a gel-like state upon administration of the formulation, thus forming a gel-like depot of benzodiazepine antagonist from which the benzodiazepine antagonist is gradually released over time. Preferably the gel-like depot forms at or near the site of administration.

The term “non-systemic administration”, as used in the present invention, means that administration is not directly into the blood circulation. Preferably the non-systemic administration is intramuscular, intradermal or subcutaneous administration.

Benzodiazepine antagonists include, for example, flumazenil, ethyl-5-isopropoxy-4-methyl-beta-carboline-3-carboxylate and the compounds disclosed in U.S. Pat. No. 4,316,839, U.S. Pat. No. 4,352,815, U.S. Pat. No. 4,346,030, U.S. Pat. No. 4,489,003, U.S. Pat. No. 4,616,010 and U.S. Pat. No. 4,775,671 (herein incorporated by reference).

Polymers include polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, polymalic acids, polyamino acids, polyvinylpyrrolidones, polyethylene glycols, polyhydroxycelluloses, chitins, chitosans and polyorthoesters, or copolymers, terpolymers and combinations and mixtures thereof.

Copolymers of polylactic acid and polyglycolic acid (poly-lactide and glyclide) spontaneously form gel-like depots when a solution of the copolymer in a water miscible organic solvent is injected into the water or biological fluids. When a biologically active agent is dissolved or dispersed in the polymer solution an implant containing the drug is formed at the site of injection and the polymer releases the drug at a sustained release rate by diffusion and/or polymer degradation.

Degradation copolymers of poly-lactide and glyclide can range from 3 weeks to over a year, depending on the composition of the copolymer as well as the method of preparation and formulation.

A water-miscible pharmacologically accepted organic solvent, such as NMP, 2-pyrrolidone, dimethyl sulfoxide, glycerol formal, ethanol, propylene glycol or a combination or mixture thereof may be used.

Release rate can be affected by copolymer composition (ratio of poly-lactide to glycolide), copolymer molecular weight, copolymer concentration, loading of drug, solvent, implant size and shape, etc.

It may be desirable to include other ingredients in the composition of the invention; for example a buffering agent, an antioxidant, a free radical scavenger, an antimicrobial agent, and/or a coloring agent. Exact formulations and methods of manufacture will be apparent to those skilled in the art. A number of texts provide assistance in the design and manufacture of pharmaceutical formulations, including Remington's Pharmaceutical Sciences, Mack Publishing Company Co., Easton, Pa.; Remington: The Science and

Practice of Pharmacy, Mack Publishing Company Co., Easton, Pa; Pharmaceutical dosage forms and drug delivery, Ansel et al, 1995, Williams and Wilkins, Malvern, Pa.; and British Pharmacopoeia, The Stationary Office, London.

### EXAMPLES

Experiments were conducted to develop an injectable sustained release system to maintain a therapeutic concentration of flumazenil for 1 to 14 days.

1. Experimental System Optimisation

Experiments were performed to optimise an experimental system for assessing flumazenil release.

1.1 Dialysis System

Materials

Dissolution apparatus: Vankel VK 7000 (Agilent Technologies Australia)

Temperature: 37° C.

Speed: 20 rpm

Dialysis bags: dialysis tubing cellulose membrane from Sigma-Aldrich Australia—average flat width 25 mm, average diameter 16 mm (when full), retains >90% of cytocrome C (M.W. 12.400) in solution over a 10 hrs period.

Methods

Dialysis tubing cellulose membrane was cut into approximately 8 cm lengths, washed with hot water for 3 hours. Tubing was then treated with 0.3% (w/v) solution of sodium sulfide at 80° C. for 1 minute, washed with hot water (60° C.) for 2 minutes and followed by acidification with 0.2% sulfuric acid. Tubing was then rinsed with hot water to remove the acid. Test formulations were added, the tubing closed with nylon closures and placed in the dialysate. At various time periods, the dialysate was sampled and the flumazenil released measured by HPLC assay.

1.2 HPLC Assay

Chromatography column: C18-250 mm×4.6 mm×5 μm (Sigma Aldrich Australia)

Mobile phase: H₂O pH 2:methanol:tetrahydrofuran (80:13:7)

Flow: 1 ml/min

UV: 230 nm

Injection volume: 10 μl and 100 μl

1.3. Effect of Dialysate Volume on Diffusion Rate

Experiments were conducted to check the influence of the volume of the dialysis medium (dialysate) on diffusion rate.

Dialysis System

As described in 1.1

Dialysate

A=800 ml of phosphate buffer pH 4.0

B=600 ml of phosphate buffer pH 4.0

HPLC Assay

As described in 1.2

Sample

0.25 g of 3% flumazenil solution in N-Methyl-2-Pyrrolidinone (NMP)

Results

% flumazenil released

| Time (hours) | A1 | A2 | B1 | B2 |
|---|---|---|---|---|
| 1 | 57.6 | 50.3 | 52.2 | 50.5 |
| 2 | 73.1 | 66.0 | 69.2 | 66.5 |
| 4 | 94.5 | 88.6 | 92.7 | 89.2 |
| 6 | 100.9 | 98.9 | 99.2 | 98.0 |
| 24 | 100.7 | 101.7 | 100.64 | 99.2 |

Conclusion

Equilibrium for A and B was reached at similar time (around 6 hours). Increasing dialysate volume does not increase diffusion rate.

1.4 Effect of Sample Size on Diffusion Rate

Experiments were conducted to test the influence of sample size on diffusion rate.

Dialysis System

As described in 1.1 with 600 ml of phosphate buffer pH 4.0 as dialysate.

HPLC Assay

As described in 1.2

Samples

A=0.125 g of a 3% flumazenil solution in NMP

B=0.250 g of a 3% flumazenil solution in NMP

C=0.500 g of a 3% flumazenil solution in NMP

Results

| | |
|---|---|
| Time | % flumazenil released |

| (hours) | A1 | A2 | B1 | B2 | C1 | C2 |
|---|---|---|---|---|---|---|
| 1 | 85.8 | 77.9 | 50.9 | 50.9 | 36.6 | 46.5 |
| 2 | 94.9 | 90.8 | 64.7 | 65.2 | 45.5 | 56.0 |
| 4 | 100.1 | 99.1 | 87.0 | 87.6 | 59.3 | 71.1 |
| 6 | 101.8 | 101.3 | 96.3 | 97.8 | 70.4 | 83.1 |
| 24 | 101.6 | 102.0 | 101.3 | 101.3 | 100.8 | 101.0 |

Conclusion

Smaller sample sizes reach equilibrium faster.

1.5. Effect of Sample Concentration on Diffusion Rate

Experiments were conducted to test the effect of sample concentration on diffusion rate.

Dialysis System

As described in 1.1 with 600 ml of phosphate buffer pH 4.0 as dialysate.

HPLC Assay

As described in 1.2

Samples

A=0.25 g of a 1.5% flumazenil solution in NMP

B=0.25 g of a 3.0% flumazenil solution in NMP

C=0.25 g of a 4.5% flumazenil solution in NMP

Results

| | |
|---|---|
| Time | % flumazenil released |

| (hours) | A1 | A2 | B1 | B2 | C1 | C2 |
|---|---|---|---|---|---|---|
| 1 | 92.9 | 89.8 | 62.1 | 62.0 | 27.5 | 29.1 |
| 2 | 100.5 | 100 | 74.8 | 76.3 | 40.4 | 43.0 |
| 3 | 101.4 | 101.4 | 83.4 | 86.9 | 52.3 | 54.9 |
| 4 | 101.2 | 101.3 | 89.9 | 92.9 | 63.4 | 64.3 |
| 5 | 101.4 | 101.0 | 94.6 | 96.6 | 72.5 | 71.7 |
| 6 | 101.3 | 101.2 | 97.9 | 99.1 | 80.7 | 78.5 |
| 24 | 100.1 | 100.0 | 99.6 | 99.2 | 100.4 | 100.3 |

Note:
no precipitation was observed in sample A during the dialysis. Precipitation was observed in samples B and C during dialysis.

Conclusion

Lower concentrations reach equilibrium faster.

1.6 Effect of Concentration and Sample Size on Diffusion Rate

Experiments were conducted to test the effect of different concentrations and different sample sizes (but same quantity of flumazenil) on diffusion rate.

Dialysis System

As described in 1.1 with 600 ml of phosphate buffer pH 4.0 as dialysate.

HPLC Assay

As described in 1.2

Samples

A=0.25 g of a 1.5% flumazenil solution in NMP

B=0.125 g of a 3% flumazenil solution in NMP

C=0.083 g of a 4.5% flumazenil solution in NMP

Results

| | |
|---|---|
| Time | % flumazenil released |

| (hours) | A1 | A2 | B1 | B2 | C1 | C2 |
|---|---|---|---|---|---|---|
| 1 | 91.0 | 93.2 | 92.3 | 80.6 | 56.1 | 43.5 |
| 2 | 100.3 | 100.7 | 99.0 | 92.4 | 79.3 | 66.1 |
| 3 | 101.0 | 100.9 | 100.4 | 97.3 | 92.3 | 82.3 |
| 4 | 100.9 | 101.0 | 101.1 | 99.3 | 98.1 | 93.6 |
| 5 | 101.0 | 100.6 | 100.9 | 100.3 | 100.1 | 99.2 |
| 6 | 100.8 | 100.7 | 101.1 | 100.6 | 100.7 | 101.2 |

Note:
precipitation was observed in bags during the dialysis: A (no precipitation observed) < B < C

Conclusion

Lower concentrations reach equilibrium faster.

2 Effect of Polymer on Flumazenil Release

Experiments were performed to determine the effect of a co-polymer of poly-lactide and glyclide on flumazenil release.

2.1 Formulations

| | Formulation 1 | Formulation 2 |
|---|---|---|
| | (0% polymer) | (35% polymer) |
| Flumazenil | 3 g | 3 g |
| DL-lactide/glycolide | 0 g | 35 g |
| NMP | qs 100 ml | qs 100 ml |

2.2 Dialysis System

As described in 1.1 with 600 ml of phosphate buffer pH 4.0 as dialysate.

2.3 HPLC Assay

As described in 1.2

2.4 Results

% flumazenil released

| Time | Formulation 1 | Formulation 2 |
|---|---|---|
| (hours) | (0% polymer) | (35% polymer) |
| 6 | 101.24 | 29.66 |
| 24 | 101.30 | 40.80 |
| 48 | 101.30 | 50.66 |
| 72 | 101.08 | 57.46 |

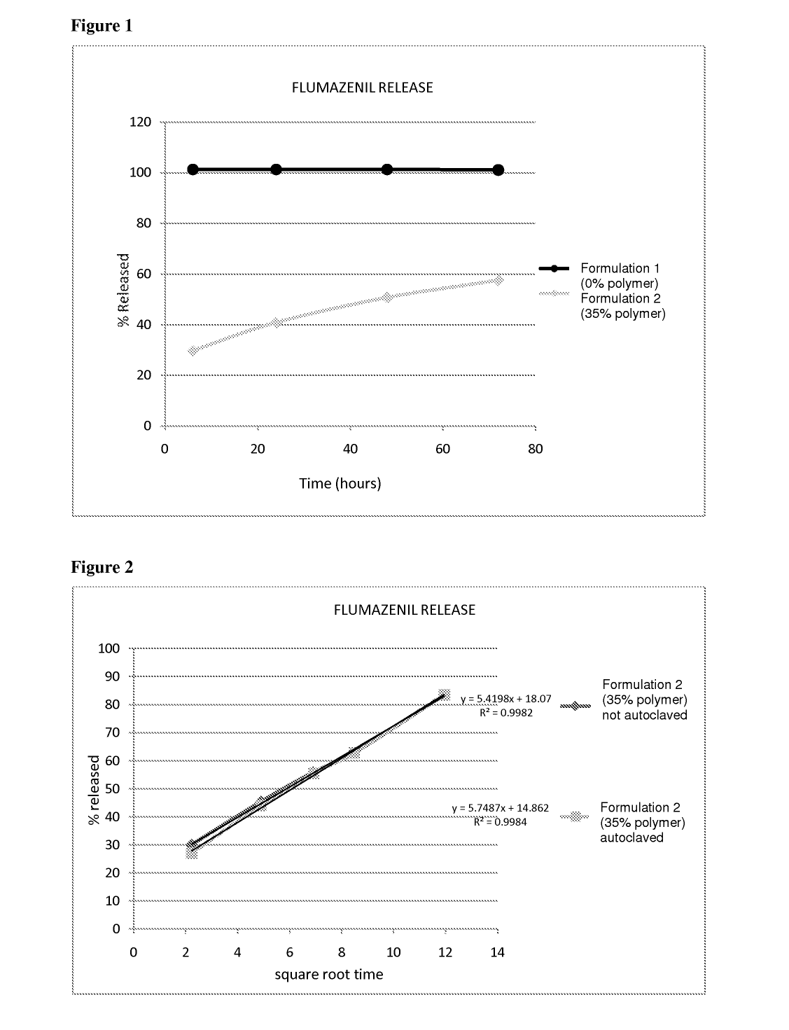

The above results are shown in FIG. 1

2.5 Conclusion

A co-polymer of poly-lactide and glyclide significantly reduce the rate of flumazenil release (i.e., delay flumazenil release).

3 Effect of Autoclaving on Flumazenil Release

Experiments were performed to determine the effect of autoclaving on flumazenil release from a co-polymer of poly-lactide and glyclide.

3.1 Formulations

| | Formulation 2 | Formulation 2 |
|---|---|---|
| | (35% polymer) | (35% polymer) |
| | not autoclaved | autoclaved |
| Flumazenil | 3 gv | 3 g |
| DL-lactide/glycolide | 35 g | 35 g |
| NMP | qs 100 ml | qs 100 ml |

3.2 Dialysis System

As described in 1.1 with 600 ml of phosphate buffer pH 4.0 as dialysate.

3.3 HPLC Assay

As described in 1.2

3.4 Results

| Time | | | | |
|---|---|---|---|---|
| (hours) | replicate 1 | replicate 2 | replicate 3 | Average |
| | Formulation 2 (35% polymer) not autoclaved | | | |
| | % flumazenil released | | | |
| 24 | 43.0 | 50.5 | 42.6 | 45.4 (9.8) |
| 48 | 52.5 | 63.0 | 52.3 | 55.9 (11.0) |
| 72 | 58.4 | 71.2 | 58.4 | 62.7 (11.8) |
| 143 | 78.0 | 91.3 | 81.0 | 83.4 (8.3) |
| | Formulation 2 (35% polymer) autoclaved | | | |
| | % flumazenil released | | | |
| 24 | 40.5 | 43.9 | 47.4 | 43.9 (7.9) |
| 48 | 49.3 | 56.3 | 60.9 | 55.5 (10.5) |
| 72 | 55.5 | 64.1 | 68.7 | 62.8 (10.7) |
| 143 | 78.6 | 84.3 | 87.7 | 83.6 (5.5) |

The above results are shown in FIG. 2

3.5 Conclusion

Autoclaving does not significantly affect flumazenil release rate from a co-polymer of poly-lactide and glyclide.

4 Effect of Polymer Concentration on Flumazenil Release

Experiments were performed to determine the effect of co-polymer of poly-lactide and glyclide concentration on flumazenil release.

4.1 Formulations

| | Formulation 3 | Formulation 4 |
|---|---|---|
| | (30% polymer) | (40% polymer) |
| Flumazenil | 3 g | 3 g |
| DL- lactide/glycolide | 30 g | 40 g |
| NMP | qs 100 ml | qs 100 ml |

4.2 Dialysis System

As described in 1.1 with 600 ml of phosphate buffer pH 4.0 as dialysate.

4.3 HPLC Assay

As described in 1.2

4.4 Results

| | Formulation 2 | Formulation 3 | Formulation 4 |
|---|---|---|---|
| Time | (35% polymer) | (30% polymer) | (40% polymer) |
| (hours) | % released | % released | % released |
| 6 | 30.6 | 42.4 | 19.1 |
| 24 | 45.5 | 57.7 | 30.4 |
| 48 | 57.6 | 67.3 | 40.7 |
| 72 | 65.7 | 73.1 | 47.9 |

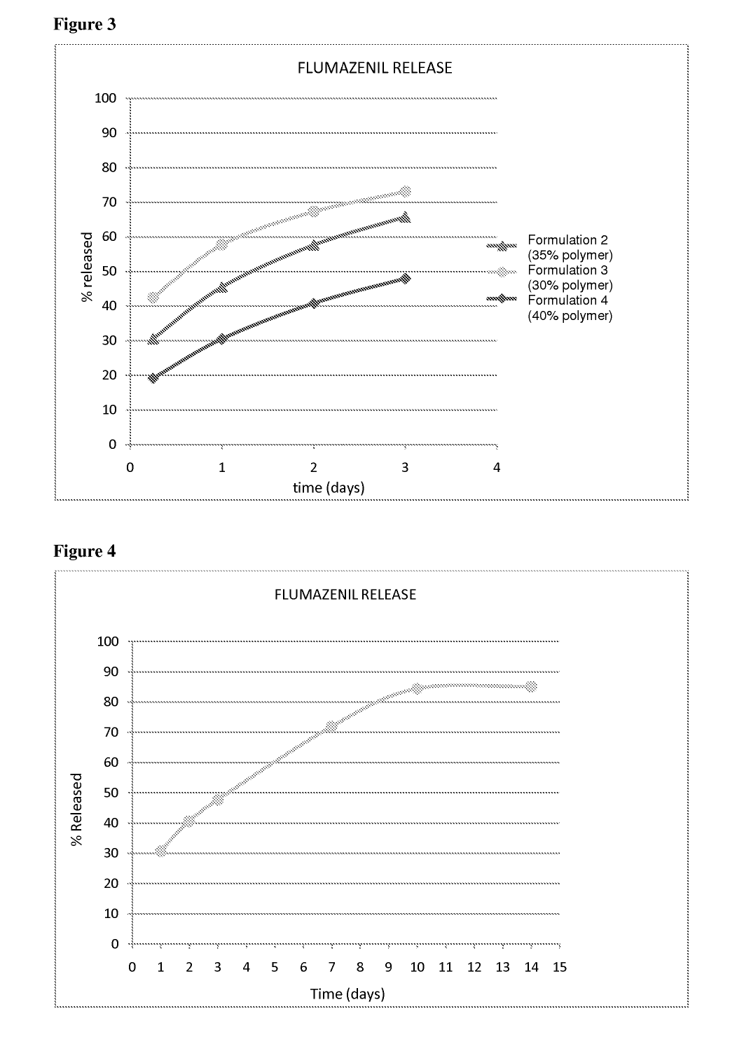

The above results are shown in FIG. 3

4.5 Conclusion

A 40% co-polymer of poly-lactide and glyclide significantly reduces the rate of flumazenil release (i.e., delay flumazenil release) compared to 30% and 35% co-polymers.

5. Flumazenil Release Over 14 Days in Vitro

In vitro experiments were performed to determine flumazenil release over 14 days from a 40% co-polymer of poly-lactide and glyclide.

5.1 Formulation

| | Formulation 4 |
|---|---|
| | (40% polymer) |
| Flumazenil | 3 g |
| DL-lactide/glycolide | 40 g |
| NMP | qs 100 ml |

5.2 Dialysis System

As described in 1.1 with 600 ml of phosphate buffer pH 4.0 as dialysate.

5.3. HPLC Assay

As described in 1.2

5.4 Results

| | Formulation 4 | Formulation 4 | Formulation 4 |
|---|---|---|---|
| | (40% polymer) | (40% polymer) | (40% polymer) |
| | replicate 1 | replicate 2 | Average |
| Days | % released | % released | % released |
| 1 | 29.3 | 32.0 | 30.7 |
| 2 | 38.3 | 42.8 | 40.5 |
| 3 | 46.3 | 49.0 | 47.6 |
| 7 | 70.1 | 73.6 | 71.8 |
| 10 | 83.5 | 85.2 | 84.4 |
| 14 | 84.3 | 85.8 | 85.0 |

The above results are shown in FIG. 4

5.5 Conclusion

A 40% co-polymer of poly-lactide and glyclide significantly reduces the rate of flumazenil release over 14 days in vitro.

6. Flumazenil Release Over 14 Days in Vivo

In vivo experiments were performed to determine flumazenil release over 14 days from a 40% co-polymer of poly-lactide and glyclide.

6.1 Formulation

| | Formulation 4 |
|---|---|
| | (40% polymer) |
| Flumazenil | 3 g |
| DL-lactide/glycolide | 40 g |
| NMP | qs 100 ml |

6.2 In Vivo Experiments

2.0 ml of Formulation 4 (representing 60mg of flumazenil) was injected intramuscularly into 12 methamphetamine dependent patients. Blood samples were taken for analysis at various time intervals over a 14 day period. Additionally, patients were monitored for methamphetamine cravings and withdrawal symptoms.

6.3 Blood Sample Preparation

Blood was centrifuged and the plasma collected and frozen until assay. For assaying, the pH of the plasma was adjusted to 3.5 to ensure the flumazenil is in its most lipophilic form. The flumazenil was then extracted from the plasma with 3 lots of a non polar solvent, such as hexane. The hexane was then evaporated and the residue reconstituted with mobile phase and analysed by HPLC.

6.4. HPLC Assay

As described in 1.2

6.5 Results

| Time post | Flumazenil in blood |
|---|---|
| administration (days) | (ng/ml) |
| 1 | 2.6 to 3.2 |
| 4 | 2.2 to 2.8 |
| 6 | 2.8 to 3.6 |
| 9 | 2.9 to 3.7 |
| 12 | 1.8 to 2.2 |
| 14 | 1.3 to 1.7 |

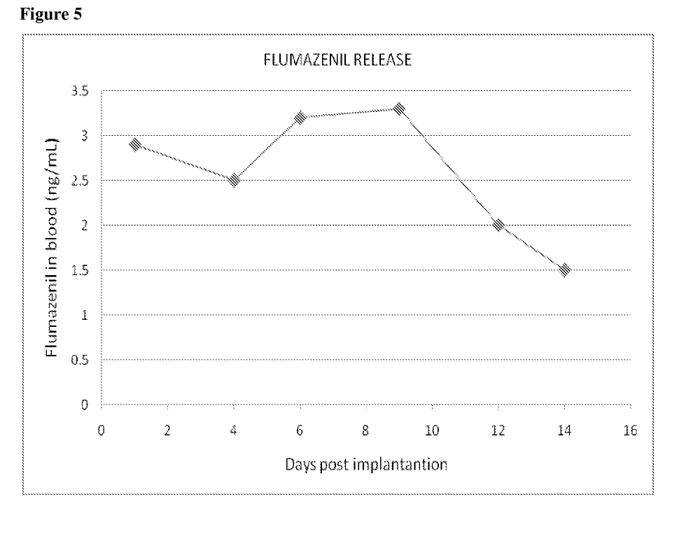

The above results are shown in FIG. 5.

11 of 12 patients exhibited no methamphetamine cravings or withdrawal symptoms during the 14-day period.

6.6 Conclusion

These results illustrate that therapeutic plasma levels of flumazenil for 14 days can be achieved by a single intramuscular injection of a formulation comprising flumazenil and a 40% co-polymer of poly-lactide and glyclide.

Although the invention has been described with reference to specific examples, it will be appreciated by those skilled in the art that the invention may be embodied in many other forms.

1. A sustained release pharmaceutical composition comprising a benzodiazepine antagonist and a biocompatible excipient.

2. The composition according to claim 1 wherein the biocompatible excipient is a polymer that is able to form a gel-like depot.

3. A sustained release pharmaceutical composition comprising a benzodiazepine antagonist and a polymer that is able to form a gel-like depot.

4. The composition according to claim 2 wherein the polymer is selected from the group consisting of polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, polymalic acids, polyamino acids, polyvinylpyrrolidones, polyethylene glycols, polyhydroxycelluloses, chitins, chitosans and polyorthoesters, and copolymers, terpolymers and combinations and mixtures thereof.

5. The composition according to claim 2 wherein the polymer is selected from the group consisting of poly lactides and copolymers thereof with glycolide.

6. The composition according to claim 1 wherein the benzodiazepine antagonist is flumazenil.

7. The composition according to claim 1 wherein the composition comprises flumazenil and a copolymer of 3,6-dimethyl-1,4-dioxane-2,S-dione and 1,4-dioxane-2,S-dione.

8. The composition according to claim 1 wherein the composition further comprises a water-miscible pharmaceutically acceptable organic solvent.

9. The composition according to claim 8 wherein the organic solvent is selected from the group consisting of N-methyl-2-pyrrolidone, 2-pynolidone, dimethyl sulfoxide, glycerol formal, ethanol, propylene glycol and combination and mixtures thereof.

10. The composition according to claim 1 wherein the composition comprises flumazenil, a copolymer of 3,6-dimethyl-1,4-dioxane-2,5-dione and 1,4-dioxane-2,5-dione, and N-methyl-2-pyrrolidone.

11. The composition according to claim 1 wherein the composition is adapted for intramuscular, intradermal or subcutaneous administration.

12. The composition according to claim 11 wherein the administration is by injection.

13. The composition according to claim 12 wherein the composition is in liquid form and forms a gel-like depot upon intramuscular, intradermal or subcutaneous injection.

14. A sustained release pharmaceutical composition comprising a benzodiazepine antagonist and a polymer that is able to form a gel-like depot, wherein the composition has the following in vitro dissolution rate when measured using a Vankel VK 7000 dissolution apparatus set at 37° C. and 20 rpm, a dialysate of 800 ml of phosphate buffer pH 4.0 and dialysis tubing that retains >90% of cytocrome C (M.W. 12.400) in solution over a 10 hrs period:
between 10 and 30% of benzodiazepine antagonist released after 6 hours,
between 20 and 40% of benzodiazepine antagonist released after 24 hours,
between 30 and 50% of benzodiazepine antagonist released after 2 days,
between 40 and 60% of benzodiazepine antagonist released after 3 days,
at least 60% of benzodiazepine antagonist released after 7 days,
at least 70% of benzodiazepine antagonist released after 10 days,
at least 80% of benzodiazepine antagonist released after 14 days.

15. The composition according to claim 14 wherein the composition comprises flumazenil and a copolymer of 3,6-dimethyl-1,4-dioxane-2,5-dione and 1,4-dioxane-2,5-dione.

16. A method for treating a disease or disorder, an addiction or dependence, or an overdose in a subject comprising administering to the subject a composition according to claim 1.

17. The method according to claim 16 wherein the disease or disorder is selected from the group consisting of sleepiness, a nervous system disorder, a psychiatric condition, Down Syndrome, a depressive disorder, memory deficit and neuropathic pain.

18. The method according to claim 16 wherein the addiction or dependence is selected from the group consisting of drug addiction, alcohol and/or stimulant substance abuse, cocaine dependence, methamphetamine dependence and alcohol dependence.

19. The method according to claim 16 wherein the addiction or dependence is methamphetamine dependence.

20. The method according to claim 16 wherein the overdose is benzodiazepine overdose.

21.-27. (canceled)
